(19) United States
(12) Patent Application Publication
(10) Pub. No.: US 2025/0195476 A1
(43) Pub. Date: Jun. 19, 2025

(54) JAK1 PATHWAY INHIBITORS FOR THE TREATMENT OF PRURIGO NODULARIS

(71) Applicant: Incyte Corporation, Wilmington, DE (US)

(72) Inventors: Paul Smith, Carlsbad, CA (US); Kurt Andrew Brown, Narberth, PA (US)

(21) Appl. No.: 19/067,059

(22) Filed: Feb. 28, 2025

Related U.S. Application Data
(63) Continuation of application No. 17/735,276, filed on May 3, 2022, now Pat. No. 12,268,667.
(60) Provisional application No. 63/183,249, filed on May 3, 2021.

(51) Int. Cl. A61K 31/4155; A61K 9/00; A61K 31/437; A61K 31/497; A61K 31/519; A61K 31/5377; A61K 39/395; A61P 17/04

(52) U.S. Cl. CPC A61K 31/4155; A61K 9/0053; A61K 31/437; A61K 31/497; A61K 31/519; A61K 31/5377; A61K 39/3955; A61P 17/04

(57) ABSTRACT

This disclosure relates to JAK1 pathway inhibitors and their use in treating prurigo nodularis.

The present application a continuation application of and claims priority to U.S. application Ser. No. 17/735,276, filed on May 3, 2022, which claims the benefit of U.S. Provisional Application No. 63/183,249, filed May 3, 2021, which is incorporated herein by reference in its entirety.

U.S. Provisional Application No. 63/183,225, filed May 3, 2021, and the U.S. Non-Provisional application Ser. No. 17/735,273, entitled “Ruxolitinib for the Treatment Of Prurigo Nodularis”, filed May 3, 2022, claiming priority to said U.S. Provisional Application No. 63/183,225, are incorporated herein by reference in their entireties.

### TECHNICAL FIELD

This disclosure relates to JAK1 pathway inhibitors and the use thereof in treating prurigo nodularis.

### BACKGROUND

Prurigo nodularis (PN) is a chronic skin disorder characterized by firm, dome-shaped, intensely pruritic nodules ranging in size from a few millimeters to several centimeters. The nodules are often symmetrically distributed on the extensor surfaces of the arms and legs and on the trunk. PN has an estimated prevalence of 72 per 100,000 persons in the US, based on people with health insurance. Reports vary on whether it more frequently in women versus men or occurs equally in men and women. People with darker or greater degrees of pigmentation in their skin are substantially more likely to have PN than patients with lighter or less pigmentation in their skin. In particular, one study found that African American patients were 3.4 times more likely to have PN than Caucasian patients.

Quality of life can be substantially negatively impacted by PN. In particular, quality of life issues include sleep disturbance, impact on job performance, and avoidance of social activities. In addition, the burden of systemic comorbidities in prurigo nodularis often exceeds that of other inflammatory skin disorders (e.g., atopic dermatitis or psoriasis). Prurigo nodularis is associated with increased rates of mental health (specifically anxiety and depression), endocrine, cardiovascular, and renal disorders, as well as HIV and malignancy. Approximately half of all patients with PN report a history of atopic dermatitis.

Pharmacologic therapy with first-generation sedating antihistamines (e.g., hydroxyzine, diphenhydramine) administered at bedtime may be useful in controlling nocturnal pruritus. Both selective serotonin reuptake inhibitors and tricyclic antidepressants are also employed for chronic pruritus, especially when a component of depression is present.

Super-potent topical corticosteroids are considered first-line therapy. Patients with widespread disease may be given phototherapy. Patients with recalcitrant PN may be given systemic treatments including systemic immunosuppressants, thalidomide, lenalidomide, and anticonvulsants. These treatments are associated with potential significant toxicity, and their efficacy in patients with recalcitrant PN has not been established. Accordingly, there is a need to develop new therapies for the treatment of prurigo nodularis. This application addresses this need and others.

### DESCRIPTION OF THE DRAWINGS

FIG. 1 depicts a representation of skin punch biopsies used for the JAK1 mediated pharmacological inhibition of PN pathophysiology.

FIG. 2 depicts a graphical representation of JAK1 mediated pharmacological inhibition of PN pathophysiology using JAK1 inhibitor Compound 1.

FIG. 3 depicts an outline of a phase 2 randomized, double-blind, placebo-controlled dose-ranging study of the efficacy and safety of Compound 1.

### SUMMARY

Provided herein are methods for the treatment of prurigo nodularis in a subject in need thereof, comprising administering to the subject a therapeutically effective amount of a JAK1 pathway inhibitor, or a pharmaceutically acceptable salt thereof.

Provided herein is a JAK1 pathway inhibitor, or a pharmaceutically acceptable salt thereof, for the treatment of prurigo nodularis in a subject in need thereof.

Provided herein is a use of a JAK1 pathway inhibitor, or a pharmaceutically acceptable salt thereof, for manufacture of a medicament for use in treating prurigo nodularis in a subject in need thereof.

### DETAILED DESCRIPTION

The present invention provides, inter alia, a method of treating prurigo nodularis in a subject in need thereof, comprising administering to the subject a therapeutically effective amount of a JAK1 pathway inhibitor, or a pharmaceutically acceptable salt thereof.

In some embodiments, the JAK1 pathway inhibitor, or a pharmaceutically acceptable salt thereof, is selective for JAK1 over JAK2, JAK3, and Tyk2.

In some embodiments, the JAK1 pathway inhibitor is 4-[3-(cyanomethyl)-3-(3′,5′-dimethyl-1H, 1′H-4,4′-bipyrazol-1-yl)azetidin-1-yl]-2,5-difluoro-N-[(1S)-2,2,2-trifluoro-1-methylethyl]benzamide (Compound 1), or a pharmaceutically acceptable salt thereof.

In some embodiments, the JAK1 pathway inhibitor is 4-[3-(cyanomethyl)-3-(3′,5′-dimethyl-1H, 1′H-4,4′-bipyrazol-1-yl)azetidin-1-yl]-2,5-difluoro-N-[(1S)-2,2,2-trifluoro-1-methylethyl]benzamide phosphoric acid salt.

In some embodiments, the JAK1 pathway inhibitor is {1-{1-[3-fluoro-2-(trifluoromethyl)isonicotinoyl]piperidin-4-yl}-3[4-(7H-pyrrolo [2,3-d]pyrimidin-4-yl)-1H-pyrazol-1-yl]azetidin-3-yl}acetonitrile, or a pharmaceutically acceptable salt thereof.

In some embodiments, the JAK1 pathway inhibitor is {1-{1-[3-fluoro-2-(trifluoromethyl)isonicotinoyl]piperidin-4-yl}-3[4-(7H-pyrrolo[2,3-d]pyrimidin-4-yl)-1H-pyrazol-1-yl]azetidin-3-yl}acetonitrile adipic acid salt.

In some embodiments, the JAK1 pathway inhibitor, or a pharmaceutically acceptable salt thereof, is administered in a daily dose of about 5 mg to about 95 mg on a free base basis.

In some embodiments, the JAK1 pathway inhibitor, or a pharmaceutically acceptable salt thereof, is administered in a daily dose of about 15 mg, about 45 mg, about 75 mg, or about 90 mg on a free base basis.

In some embodiments, the JAK1 pathway inhibitor, or pharmaceutically acceptable salt thereof, is administered in a daily dose of about 45 mg or about 75 mg on a free base basis.

In some embodiments, the JAK1 pathway inhibitor, or pharmaceutically acceptable salt thereof, is administered in combination with a further therapeutic agent.

In some embodiments, the further therapeutic agent is a neurokinin 1 receptor antagonist. In some embodiments, the neurokinin 1 receptor antagonist is aprepitant.

In some embodiments, the further therapeutic agent is anti-IL-4/IL-13 antibody. In some embodiments, the anti-IL-4/IL-13 antibody is dupilumab, lebrikizumab or tralokinumab.

In some embodiments, the further therapeutic agent is an anti-IL-5 antibody. In some embodiments, the anti-IL-5 antibody is benralizumab, mepolizumab, or reslizumab.

In some embodiments, the further therapeutic agent is an anti-IL-31 antibody. In some embodiments, the anti-IL-31 antibody is nemolizumab.

In some embodiments, the administering comprises administering the JAK1 pathway inhibitor, or pharmaceutically acceptable salt thereof, together with at least one pharmaceutically acceptable carrier or excipient.

In some embodiments, efficacy of the treatment method disclosed herein can be established based upon an Investigator's Global Assessment (IGA). In some embodiments, the IGA-TS (Investigator's Global Assessment Treatment Success) is defined as an IGA score of 0 or 1 with ≥2 grade improvement from baseline. Efficacy may be established if by evaluating a proportion of subjects achieving an IGA-TS (IGA of 0 or 1 with a 2-point decrease) at a designated point of time (e.g., week 16).

In some embodiments, efficacy of the treatment method disclosed herein can be established based upon Itch Numerical Rating Scale (Itch NRS). In some embodiments, efficacy may be demonstrated by achieving a preset proportion of subjects achieving at least 2 or 4-point improvement in Itch NRS (e.g., at Week 16). In some embodiments, efficacy may be demonstrated by observing time to ≥2-point or ≥4-point improvement from baseline in Itch NRS. In some embodiments, a JAK1 inhibitor (e.g., Compound 1), and/or methods of use described herein result in an improvement in a subject's response to Itch NRS from baseline. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% improvement in a subject's response to Itch NRS from baseline.

In some embodiments, efficacy of the treatment method disclosed herein can be established based upon Peak Pruritus NRS. The Peak Pruritus Numerical Rating Scale (NRS) was developed to assess one parameter or dimension of itch in clinical trials of drugs in development for patients with moderate-to-severe AD. The Peak Pruritus NRS is a patient-reported outcome test designed to measure peak pruritus, or ‘worst’ itch, over the previous 24 h. The test is based on, for example, the following question: ‘On a scale of 0 to 10, with 0 being “no itch” and 10 being “worst itch imaginable”, how would you rate your itch at the worst moment during the previous 24 hours?’ In some embodiments, efficacy may be demonstrated by achieving a preset proportion of subjects achieving at least 2 or 4-point improvement in Peak Pruritus NRS (e.g., at Week 16). In some embodiments, efficacy may be demonstrated by observing time to ≥2-point or ≥4-point improvement from baseline in Peak Pruritus NRS. In some embodiments, a JAK1 inhibitor (e.g., Compound 1), and/or methods of use described herein result in an improvement in a subject's response to Peak Pruritus NRS from baseline. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% improvement in a subject's response to Peak Pruritus NRS from baseline.

In some embodiments, efficacy of the treatment method disclosed herein can be established based upon Nodule Assessment. In some embodiments, a JAK1 inhibitor (e.g., Compound 1), and/or methods of use described herein result in an improvement in a subject's response to PAS from baseline. In some embodiments, a JAK1 inhibitor (e.g., Compound 1), and/or methods of use described herein result in about a 5%, about a 10%, about a 20%, about a 30%, about a 40%, about a 50%, about a 60%, about a 70%, about a 80%, about a 90%, or about a 95% improvement in a subject's response to PAS from baseline. In some embodiments, efficacy of the treatment method disclosed herein can be established based upon photography of effected areas. Photography of the body areas affected with PN will be obtained at visits. All sites will use 2-dimensional photography to photograph areas of the body containing PN nodules.

In some embodiments, efficacy of the treatment method disclosed herein can be established based upon patient-reported outcomes (PROs). In some embodiments, efficacy of the treatment method disclosed herein can be established based upon a Dermatology Life Quality Index (DLQI). In some embodiments, a JAK1 inhibitor (e.g., Compound 1), and/or methods of use described herein result in an improvement in a subject's response to DLQI from baseline. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% improvement in a subject's response to DLQI from baseline.

In some embodiments, efficacy of the treatment method disclosed herein can be established based upon Patient Global Impression of Change (PGIC). In some embodiments, a JAK1 inhibitor (e.g., Compound 1), and/or methods of use described herein result in an improvement in a subject's response to PGIC from baseline. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% improvement in a subject's response to PGIC from baseline.

In some embodiments, efficacy can be evaluated based upon improvement in a subject's Hospital Anxiety and Depression Scale (HADS). In some embodiments, a JAK1 inhibitor (e.g.,

Compound 1), and/or methods of use described herein result in an improvement in a subject's response to HADS from baseline. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% improvement in a subject's response to HADS from baseline.

In some embodiments, efficacy can be evaluated based upon improvement in a subject's Functional Assessment of Chronic Illness Therapy—Fatigue Scale (FACIT—Fatigue Scale). In some embodiments, a JAK1 inhibitor (e.g., Compound 1), and/or methods of use described herein result in an improvement in a subject's response to FACIT-Fatigue Scale from baseline. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% improvement in a subject's response to FACIT-Fatigue Scale from baseline.

In some embodiments, efficacy can be evaluated based upon improvement in a subject's PROMIS Sleep Scale. In some embodiments, a JAK1 inhibitor (e.g., Compound 1), and/or methods of use described herein result in an improvement in a subject's response to PROMIS Sleep Scale from baseline. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% improvement in a subject's response to PROMIS Sleep Scale from baseline.

In some embodiments, efficacy can be evaluated based upon improvement in a subject's EQ-5D-5L questionnaire. In some embodiments, a JAK1 inhibitor, e.g., Compound 1, and/or methods of use described herein result in an improvement in a subject's response to the EQ-5D-5L questionnaire from baseline. In some embodiments, a JAK1 inhibitor, e.g., Compound 1, and/or methods of use described herein result in about a 5%, about a 10%, about a 20%, about a 30%, about a 40%, about a 50%, about a 60%, about a 70%, about a 80%, about a 90%, or about a 95% improvement in a subject's response to EQ-5D-5L from baseline.

The methods described herein utilize JAK1 pathway inhibitors, particularly JAK1 selective inhibitors. A JAK1 selective inhibitor is a compound that inhibits JAK1 activity preferentially over other Janus kinases. JAK1 plays a central role in a number of cytokine and growth factor signaling pathways that, when dysregulated, can result in or contribute to disease states. In autoimmune diseases and cancers, elevated systemic levels of inflammatory cytokines that activate JAK1 may also contribute to the disease and/or associated symptoms. Therefore, patients with autoimmune associated diseases like prurigo nodularis may benefit from JAK1 inhibition. Selective inhibitors of JAK1 may be efficacious while avoiding unnecessary and potentially undesirable effects of inhibiting other JAK kinases. Activated T cells have shown increased IL-22 cytokine expression in patients with PN compared with healthy controls (Belzberg, et al., J Invest Dermatol, 141 (9): 2208-2218.e14 (2021)).

In some embodiments, the JAK1 pathway inhibitor, or a pharmaceutically acceptable salt thereof, is selective for JAK1 over JAK2, JAK3, and TYK2 (i.e., a JAK1 selective inhibitor). For example, the compounds described herein, or pharmaceutically acceptable salts thereof, preferentially inhibit JAK1 over one or more of JAK2, JAK3, and TYK2. In some embodiments, the compounds inhibit JAK1 preferentially over JAK2 (e.g., have a JAK2/JAK1 IC₅₀ ratio >1). In some embodiments, the compounds or salts are about 10-fold more selective for JAK1 over JAK2. In some embodiments, the compounds or salts are about 3-fold, about 5-fold, about 10-fold, about 15-fold, or about 20-fold more selective for JAK1 over JAK2 as calculated by measuring IC₅₀ at 1 mM ATP (e.g., see Example A).

In some embodiments, the JAK1 pathway inhibitor is a compound of Table 1, or a pharmaceutically acceptable salt thereof. The compounds in Table 1 are selective JAK1 inhibitors (selective over JAK2, JAK3, and TYK2). The IC₅₀ values obtained by the method of Example A at 1 mM ATP are shown in Table 1.

The compounds of Table 1 can be prepared by the synthetic procedures described, for example, in US Patent Publ. No. 2011/0224190, filed Mar. 9, 2011, US Patent Publ. No. 2014/0343030, filed May 16, 2014, US Patent Publ. No. 2014/0121198, filed Oct. 31, 2013, US Patent Publ. No. 2010/0298334, filed May 21, 2010, US Patent Publ. No. 2011/0059951, filed Aug. 31, 2010, US Patent Publ. No. 2012/0149681, filed Nov. 18, 2011, US Patent Publ. No. 2012/0149682, filed Nov. 18, 2011, US Patent Publ. 2013/0018034, filed Jun. 19, 2012, US Patent Publ. No. 2013/0045963, filed Aug. 17, 2012, and US Patent Publ. No. 2014/0005166, filed May 17, 2013, each of which is incorporated herein by reference in its entirety.

TABLE 1

| | | | | JAK1 | |
|---|---|---|---|---|---|
| Comp. | | | | IC₅₀ | JAK2/ |
| No. | Prep. | Name | Structure | (nM) | JAK1 |
| 1 | US 2014/0343030 (Example 7) | 4-[3-(cyanomethyl)-3-(3′,5′- dimethyl-1H,1′H-4,4′- bipyrazol-1-yl)azetidin-1- yl]-2,5-difluoro-N-[(1S)- 2,2,2-trifluoro-1- methylethyl]benzamide | 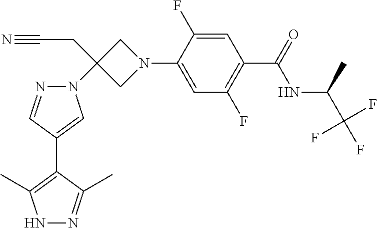 [SMILES: Cc1n[nH]c(C)c1-c1cnn(C2(CC#N)CN(c3cc(F)c(C(=O)N[C@@H](C)C(F)(F)F)cc3F)C2)c1] | +++ | >10 |
| 2 | US 2011/ 0224190 (Example 154) | 4-{3-(Cyanomethyl)-3-[4- (7H-pyrrolo[2,3- d]pyrimidin-4-yl)-1H- pyrazol-1-yl]azetidin-1-yl}- N-[4-fluoro-2- (trifluoromethyl)phenyl] piperidine-1-carboxamide | 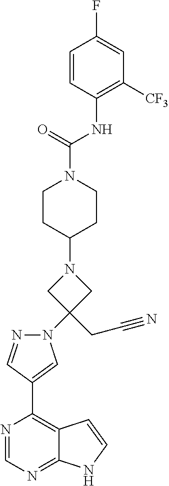 [SMILES: N#CCC1(n2cc(-c3ncnc4[nH]ccc34)cn2)CN(C2CCN(C(=O)Nc3ccc(F)cc3C(F)(F)F)CC2)C1] | + | >10 |
| 3 | US 2011/ 0224190 (Example 85) | [3-[4-(7H-pyrrolo[2,3- d]pyrimidin-4-yl)-1H- pyrazol-1-yl]-1-(1-{[2- (trifluoromethyl)pyrimidin- 4-yl]carbonyl}piperidin-4- yl)azetidin-3-yl]acetonitrile | 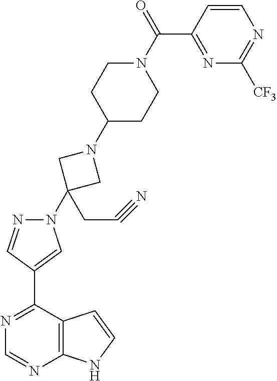 [SMILES: N#CCC1(n2cc(-c3ncnc4[nH]ccc34)cn2)CN(C2CCN(C(=O)c3ccnc(C(F)(F)F)n3)CC2)C1] | + | >10 |
| 4 | US 2011/ 0224190 (Example 1) | {1-{1-[3-Fluoro-2- (trifluoromethyl)isonicotino yl]piperidin-4-yl}-3-[4- (7H-pyrrolo[2,3- d]pyrimidin-4-yl)-1H- pyrazol-1-yl]azetidin-3- yl}acetonitrile | 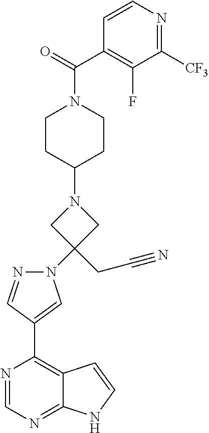 [SMILES: N#CCC1(n2cc(-c3ncnc4[nH]ccc34)cn2)CN(C2CCN(C(=O)c3ccnc(C(F)(F)F)c3F)CC2)C1] | + | >10 |
| 5 | US 2014/0121198 (Example 20) | ((2R,5S)-5-{2-[(1R)-1- hydroxyethyl]-1H- imidazo[4,5-d]thieno[3,2- b]pyridin-1-yl}tetrahydro- 2H-pyran-2-yl)acetonitrile | 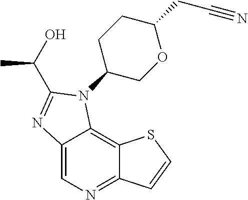 [SMILES: C[C@@H](O)c1nc2cnc3ccsc3c2n1[C@H]1CC[C@H](CC#N)OC1] | ++ | >10 |
| 6 | US 2010/ 0298334 (Example 2)^{a} | 3-[1-(6-chloropyridin-2- yl)pyrrolidin-3-yl]-3-[4- (7H-pyrrolo[2,3- d]pyrimidin-4-yl)-1H- pyrazol-1-yl]propanenitrile | 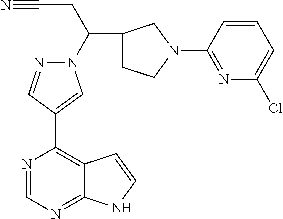 [SMILES: N#CCC(C1CCN(c2cccc(Cl)n2)C1)n1cc(-c2ncnc3[nH]ccc23)cn1] | + | >10 |
| 7 | US 2010/ 0298334 (Example 13c) | 3-(1-[1,3]oxazolo[5,4- b]pyridin-2-ylpyrrolidin-3- yl)-3-[4-(7H-pyrrolo[2,3- d]pyrimidin-4-yl)-1H- pyrazol-1-yl]propanenitrile | 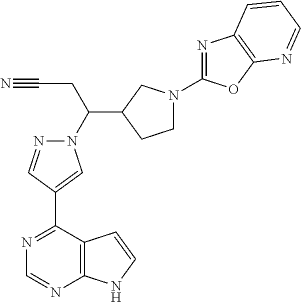 [SMILES: N#CCC(C1CCN(c2nc3cccnc3o2)C1)n1cc(-c2ncnc3[nH]ccc23)cn1] | + | >10 |
| 8 | US 2011/ 0059951 (Example 12) | 4-[(4-{3-cyano-2-[4-(7H- pyrrolo[2,3-d]pyrimidin-4- yl)-1H-pyrazol-1- yl]propyl}piperazin-1- yl)carbonyl]-3- fluorobenzonitrile | 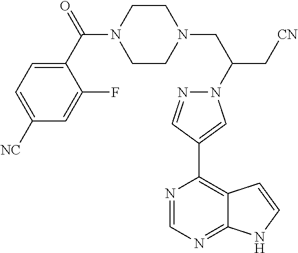 [SMILES: N#CCC(CN1CCN(C(=O)c2ccc(C#N)cc2F)CC1)n1cc(-c2ncnc3[nH]ccc23)cn1] | + | >10 |
| 9 | US 2011/ 0059951 (Example 13) | 4-[(4-{3-cyano-2-[3-(7H- pyrrolo[2,3-d]pyrimidin-4- yl)-1H-pyrrol-1- yl]propyl}piperazin-1- yl)carbonyl]-3- fluorobenzonitrile | 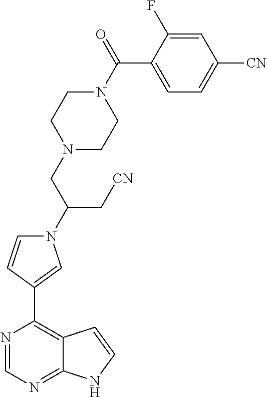 [SMILES: N#CCC(CN1CCN(C(=O)c2ccc(C#N)cc2F)CC1)n1ccc(-c2ncnc3[nH]ccc23)c1] | + | >10 |
| 10 | US 2012/ 0149681 (Example 7b) | [trans-1-[4-(7H- pyrrolo[2,3-d]pyrimidin-4- yl)-1H-pyrazol-1-yl]-3-(4- {[2- (trifluoromethyl)pyrimidin- 4-yl]carbonyl}piperazin-1- yl)cyclobutyl]acetonitrile | 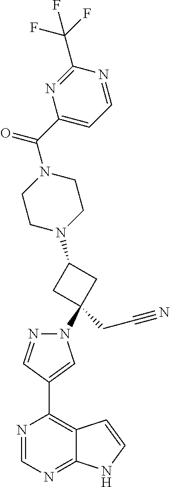 [SMILES: N#CC[C@]1(n2cc(-c3ncnc4[nH]ccc34)cn2)C[C@H](N2CCN(C(=O)c3ccnc(C(F)(F)F)n3)CC2)C1] | + | >10 |
| 11 | US 2012/ 0149681 (Example 157) | {trans-3-(4-{[4-[(3- hydroxyazetidin-1- yl)methyl]-6- (trifluoromethyl)pyridin-2- yl]oxy}piperidin-1-yl)-1- [4-(7H-pyrrolo[2,3- d]pyrimidin-4-yl)-1H- pyrazol-1- yl]cyclobutyl}acetonitrile | 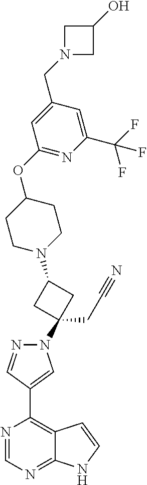 [SMILES: N#CC[C@]1(n2cc(-c3ncnc4[nH]ccc34)cn2)C[C@H](N2CCC(Oc3cc(CN4CC(O)C4)cc(C(F)(F)F)n3)CC2)C1] | + | >10 |
| 12 | US 2012/ 0149681 (Example 161) | {trans-3-(4-{[4-{[(2S)-2- (hydroxymethyl)pyrrolidin- 1-yl]methyl}-6- (trifluoromethyl)pyridin-2- yl]oxy}piperidin-1-yl)-1- [4-(7H-pyrrolo[2,3- d]pyrimidin-4-yl)-1H- pyrazol-1- yl]cyclobutyl}acetonitrile | 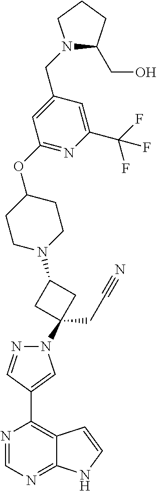 [SMILES: N#CC[C@]1(n2cc(-c3ncnc4[nH]ccc34)cn2)C[C@H](N2CCC(Oc3cc(CN4CCC[C@H]4CO)cc(C(F)(F)F)n3)CC2)C1] | + | >10 |
| 13 | US 2012/ 0149681 (Example 162) | {trans-3-(4-{[4-{[(2R)-2- (hydroxymethyl)pyrrolidin- 1-yl]methyl}-6- (trifluoromethyl)pyridin-2- yl]oxy}piperidin-1-yl)-1- [4-(7H-pyrrolo[2,3- d]pyrimidin-4-yl)-1H- pyrazol-1- yl]cyclobutyl}acetonitrile | 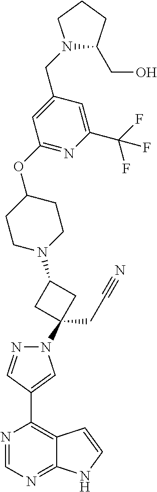 [SMILES: N#CC[C@]1(n2cc(-c3ncnc4[nH]ccc34)cn2)C[C@H](N2CCC(Oc3cc(CN4CCC[C@@H]4CO)cc(C(F)(F)F)n3)CC2)C1] | + | >10 |
| 14 | US 2012/ 0149682 (Example 20)^{b} | 4-(4-{3- [(dimethylamino)methyl]- 5-fluorophenoxy}piperidin- 1-yl)-3-[4-(7H-pyrrolo[2,3- d]pyrimidin-4-yl)-1H- pyrazol-1-yl]butanenitrile | 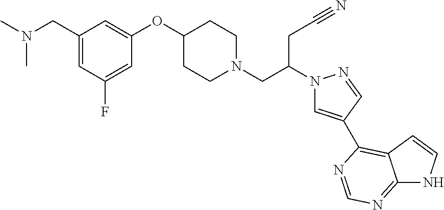 [SMILES: CN(C)Cc1cc(F)cc(OC2CCN(CC(CC#N)n3cc(-c4ncnc5[nH]ccc45)cn3)CC2)c1] | + | >10 |
| 15 | US 2013/ 0018034 (Example 18) | 5-{3-(cyanomethyl)-3-[4- (7H-pyrrolo[2,3- d]pyrimidin-4-yl)-1H- pyrazol-1-yl]azetidin-1-yl}- N-isopropylpyrazine-2- carboxamide | 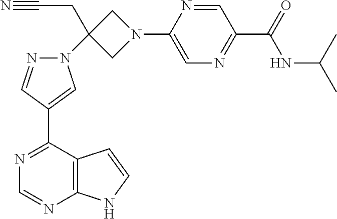 [SMILES: CC(C)NC(=O)c1cnc(N2CC(CC#N)(n3cc(-c4ncnc5[nH]ccc45)cn3)C2)cn1] | + | >10 |
| 16 | US 2013/ 0018034 (Example 28) | 4-{3-(cyanomethyl)-3-[4- (7H-pyrrolo[2,3- d]pyrimidin-4-yl)-1H- pyrazol-1-yl]azetidin-1-yl}- 2,5-difluoro-N-[(1S)-2,2,2- trifluoro-1- methylethyl]benzamide | 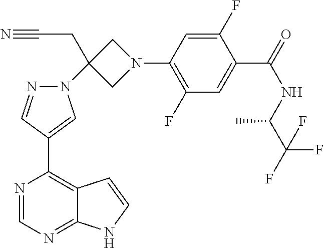 [SMILES: C[C@H](NC(=O)c1cc(F)c(N2CC(CC#N)(n3cc(-c4ncnc5[nH]ccc45)cn3)C2)cc1F)C(F)(F)F] | + | >10 |
| 17 | US 2013/ 0018034 (Example 34) | 5-{3-(cyanomethyl)-3-[4- (1H-pyrrolo[2,3-b]pyridin- 4-yl)-1H-pyrazol-1- yl]azetidin-1-yl}-N- isopropylpyrazine-2- carboxamide | 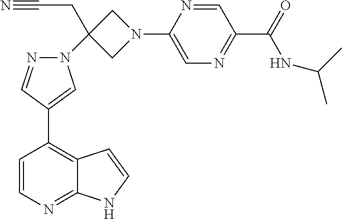 [SMILES: CC(C)NC(=O)c1cnc(N2CC(CC#N)(n3cc(-c4ccnc5[nH]ccc45)cn3)C2)cn1] | + | >10 |
| 18 | US 2013/ 0045963 (Example 45) | {1-(cis-4-{[6-(2- hydroxyethyl)-2- (trifluoromethyl)pyrimidin- 4-yl]oxy}cyclohexyl)-3-[4- (7H-pyrrolo[2,3- d]pyrimidin-4-yl)-1H- pyrazol-1-yl]azetidin-3- yl}acetonitrile | 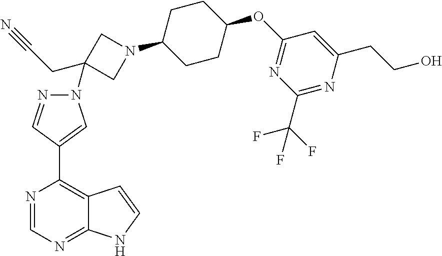 [SMILES: N#CCC1(n2cc(-c3ncnc4[nH]ccc34)cn2)CN([C@H]2CC[C@@H](Oc3cc(CCO)nc(C(F)(F)F)n3)CC2)C1] | + | >10 |
| 19 | US 2013/ 0045963 (Example 65) | {1-(cis-4-{[4- [(ethylamino)methyl]-6- (trifluoromethyl)pyridin-2- yl]oxy}cyclohexyl)-3-[4- (7H-pyrrolo[2,3- d]pyrimidin-4-yl)-1H- pyrazol-1-yl]azetidin-3- yl}acetonitrile | 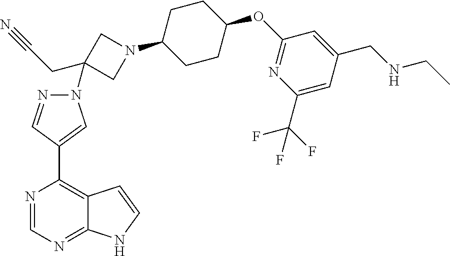 [SMILES: CCNCc1cc(O[C@H]2CC[C@@H](N3CC(CC#N)(n4cc(-c5ncnc6[nH]ccc56)cn4)C3)CC2)nc(C(F)(F)F)c1] | + | >10 |
| 20 | US 2013/ 0045963 (Example 69) | {1-(cis-4-{[4-(1-hydroxy-1- methylethyl)-6- (trifluoromethyl)pyridin-2- yl]oxy}cyclohexyl)-3-[4- (7H-pyrrolo[2,3- d]pyrimidin-4-yl)-1H- pyrazol-1-yl]azetidin-3- yl}acetonitrile | 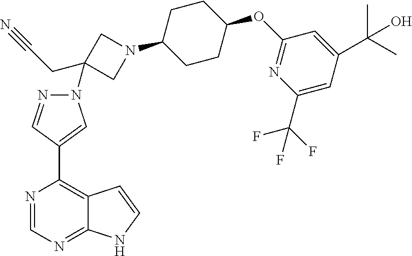 [SMILES: CC(C)(O)c1cc(O[C@H]2CC[C@@H](N3CC(CC#N)(n4cc(-c5ncnc6[nH]ccc56)cn4)C3)CC2)nc(C(F)(F)F)c1] | + | >10 |
| 21 | US 2013/ 0045963 (Example 95) | {1-(cis-4-{[4-{[(3R)-3- hydroxypyrrolidin-1- yl]methyl}-6- (trifluoromethyl)pyridin-2- yl]oxy}cyclohexyl)-3-[4- (7H-pyrrolo[2,3- d]pyrimidin-4-yl)-1H- pyrazol-1-yl]azetidin-3- yl}acetonitrile | 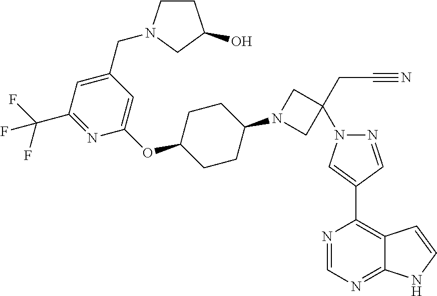 [SMILES: N#CCC1(n2cc(-c3ncnc4[nH]ccc34)cn2)CN([C@H]2CC[C@@H](Oc3cc(CN4CC[C@@H](O)C4)cc(C(F)(F)F)n3)CC2)C1] | + | >10 |
| 22 | US 2013/ 0045963 (Example 95) | {1-(cis-4-{[4-{[(3S)-3- hydroxypyrrolidin-1- yl]methyl}-6- (trifluoromethyl)pyridin-2- yl]oxy}cyclohexyl)-3-[4- (7H-pyrrolo[2,3- d]pyrimidin-4-yl)-1H- pyrazol-1-yl]azetidin-3- yl}acetonitrile | 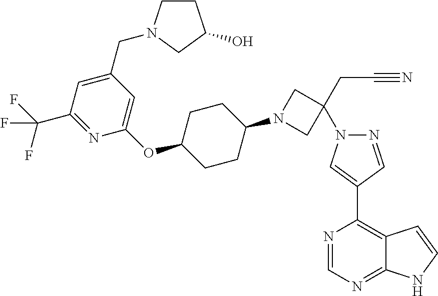 [SMILES: N#CCC1(n2cc(-c3ncnc4[nH]ccc34)cn2)CN([C@H]2CC[C@@H](Oc3cc(CN4CC[C@H](O)C4)cc(C(F)(F)F)n3)CC2)C1] | + | >10 |
| 23 | US 2014/ 0005166 (Example 1) | {trans-3-(4-{[4-({[(1S)-2- hydroxy-1- methylethyl]amino}methyl)- 6-(trifluoromethyl)pyridin- 2-yl]oxy}piperidin-1-yl)-1- [4-(7H-pyrrolo[2,3- d]pyrimidin-4-yl)-1H- pyrazol-1- yl]cyclobutyl}acetonitrile | 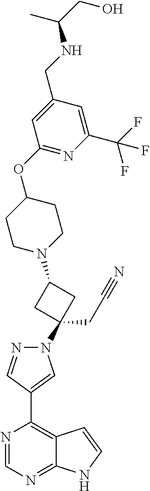 [SMILES: C[C@@H](CO)NCc1cc(OC2CCN([C@H]3C[C@](CC#N)(n4cc(-c5ncnc6[nH]ccc56)cn4)C3)CC2)nc(C(F)(F)F)c1] | + | >10 |
| 24 | US 2014/ 0005166 (Example 14) | {trans-3-(4-{[4-({[(2R)-2- hydroxypropyl]amino} methyl)-6- (trifluoromethyl)pyridin-2- yl]oxy}piperidin-1-yl)-1- [4-(7H-pyrrolo[2,3- d]pyrimidin-4-yl)-1H- pyrazol-1- yl]cyclobutyl}acetonitrile | 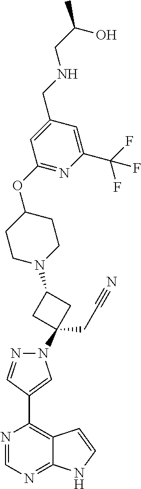 [SMILES: C[C@@H](O)CNCc1cc(OC2CCN([C@H]3C[C@](CC#N)(n4cc(-c5ncnc6[nH]ccc56)cn4)C3)CC2)nc(C(F)(F)F)c1] | + | >10 |
| 25 | US 2014/ 0005166 (Example 15) | {trans-3-(4-{[4-({[(2S)-2- hydroxypropyl]amino} methyl)-6- (trifluoromethyl)pyridin-2- yl]oxy}piperidin-1-yl)-1- [4-(7H-pyrrolo[2,3- d]pyrimidin-4-yl)-1H- pyrazol-1- yl]cyclobutyl}acetonitrile | 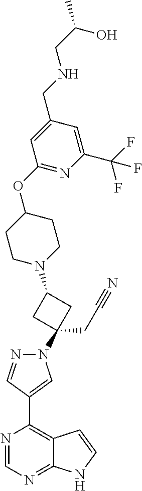 [SMILES: C[C@H](O)CNCc1cc(OC2CCN([C@H]3C[C@](CC#N)(n4cc(-c5ncnc6[nH]ccc56)cn4)C3)CC2)nc(C(F)(F)F)c1] | + | >10 |
| 26 | US 2014/ 0005166 (Example 20) | {trans-3-(4-{[4-(2- hydroxyethyl)-6- (trifluoromethyl)pyridin-2- yl]oxy}piperidin-1-yl)-1- [4-(7H-pyrrolo[2,3- d]pyrimidin-4-yl)-1H- pyrazol-1- yl]cyclobutyl}acetonitrile | 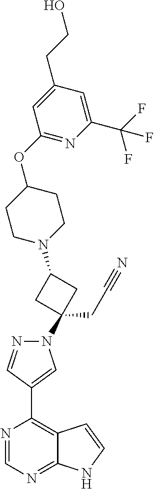 [SMILES: N#CC[C@]1(n2cc(-c3ncnc4[nH]ccc34)cn2)C[C@H](N2CCC(Oc3cc(CCO)cc(C(F)(F)F)n3)CC2)C1] | + | >10 |

+ means <10 nM (see Example A for assay conditions)
++ means ≤100 nM (see Example A for assay conditions)
+++ means ≤300 nM (see Example A for assay conditions)
^{a}Data for enantiomer 1
^{b}Data for enantiomer 2

In some embodiments, the JAK1 pathway inhibitor is 4-[3-(cyanomethyl)-3-(3′,5′-dimethyl-1H, 1′H-4,4′-bipyrazol-1-yl) azetidin-1-yl]-2,5-difluoro-N-[(1S)-2,2,2-trifluoro-1-methylethyl]benzamide (Compound 1), or a pharmaceutically acceptable salt thereof. In some embodiments, the JAK1 pathway inhibitor is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide phosphoric acid salt. Compound 1, and its salts, can be made by the procedures described in, e.g., U.S. Pat. No. 9,382,231 (see, e.g., Example 7), filed May 16, 2014, which is incorporated herein by reference in its entirety.

In some embodiments, the JAK1 pathway inhibitor is selected from the compounds, or pharmaceutically acceptable salts thereof, described in US Patent Publ. No. 2011/0224190, filed Mar. 9, 2011, US Patent Publ. No. 2014/0343030, filed May 16, 2014, US Patent Publ. No. 2014/0121198, filed Oct. 31, 2013, US Patent Publ. No. 2010/0298334, filed May 21, 2010, US Patent Publ. No. 2011/0059951, filed Aug. 31, 2010, US Patent Publ. No. 2012/0149681, filed Nov. 18, 2011, US Patent Publ. No. 2012/0149682, filed Nov. 18, 2011, US Patent Publ. 2013/0018034, filed Jun. 19, 2012, US Patent Publ. No. 2013/0045963, filed Aug. 17, 2012, and US Patent Publ. No. 2014/0005166, filed May 17, 2013, each of which is incorporated herein by reference in its entirety.

In some embodiments, the JAK1 pathway inhibitor is a compound of Formula I

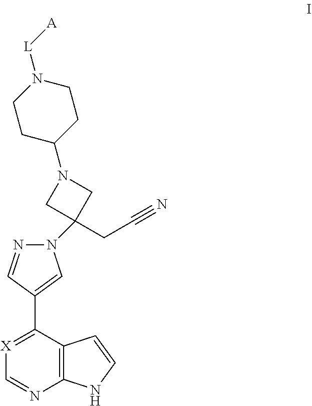

or a pharmaceutically acceptable salt thereof, wherein:
X is N or CH;
L is C(═O) or C(═O)NH;
A is phenyl, pyridinyl, or pyrimidinyl each of which is optionally substituted with 1 or 2 independently selected R¹ groups; and
each R¹ is, independently, fluoro, or trifluoromethyl.

In some embodiments, the compound of Formula I is {1-{1-[3-fluoro-2-(trifluoromethyl)isonicotinoyl]piperidin-4-yl}-3[4-(7H-pyrrolo [2,3-d]pyrimidin-4-yl)-1H-pyrazol-1-yl]azetidin-3-yl}acetonitrile, or a pharmaceutically acceptable salt thereof.

In some embodiments, the compound of Formula I is 4-{3-(Cyanomethyl)-3-[4-(7H-pyrrolo[2,3-d]pyrimidin-4-yl)-1H-pyrazol-1-yl]azetidin-1-yl}-N-[4-fluoro-2-(trifluoromethyl)phenyl]piperidine-1-carboxamide, or a pharmaceutically acceptable salt thereof.

In some embodiments, the compound of Formula I is [3-[4-(7H-pyrrolo[2,3-d]pyrimidin-4-yl)-1H-pyrazol-1-yl]-1-(1-{[2-(trifluoromethyl) pyrimidin-4-yl]carbonyl}piperidin-4-yl) azetidin-3-yl]acetonitrile, or a pharmaceutically acceptable salt thereof.

In some embodiments, the JAK1 pathway inhibitor is a compound of Formula II

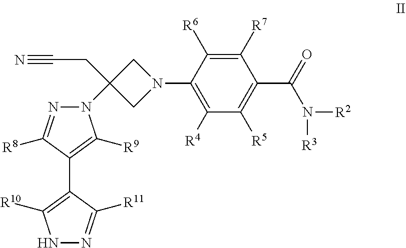

or a pharmaceutically acceptable salt thereof, wherein:
R² is C₁₋₆alkyl, C₁₋₆haloalkyl, C₃₋₆cycloalkyl, or C₃₋₆cycloalkyl-C₁₋₃alkyl, wherein said C₁₋₆alkyl, C₃₋₆cycloalkyl, and C₃₋₆cycloalkyl-C₁₋₃alkyl, are each optionally substituted with 1, 2, or 3 substituents independently selected from fluoro, —CF₃, and methyl;
R³ is H or methyl;
R⁴ is H, F, or Cl;
R⁵ is H or F;
R⁶ is H or F;
R⁷ is H or F;
R⁸ is H or methyl;
R⁹ is H or methyl;
R¹⁰ is H or methyl; and
R¹¹ is H or methyl.

In some embodiments, the JAK1 pathway inhibitor is a compound of Formula III

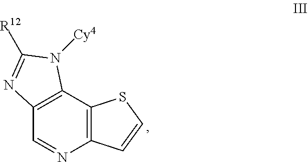

or a pharmaceutically acceptable salt thereof, wherein:
Cy⁴ is a tetrahydro-2H-pyran ring, which is optionally substituted with 1 or 2 groups independently selected from CN, OH, F, Cl, C₁₋₃alkyl, C₁₋₃haloalkyl, cyano-C₁₋₃alkyl, HO—C₁₋₃alkyl, amino, C₁₋₃alkylamino, and di(C₁₋₃alkyl) amino, wherein said C₁₋₃alkyl and di(C₁₋₃alkyl) amino is optionally substituted with 1, 2, or 3 substituents independently selected from F, Cl, C₁₋₃alkylaminosulfonyl, and C₁₋₃alkylsulfonyl; and
R¹² is —CH₂—OH, —CH(CH₃)—OH, or —CH₂—NHSO₂CH₃.

In some embodiments, the compound of Formula III is ((2R,5S)-5-{2-[(1R)-1-hydroxyethyl]-1H-imidazo [4,5-d]thieno[3,2-b]pyridin-1-yl}tetrahydro-2H-pyran-2-yl)acetonitrile, or a pharmaceutically acceptable salt thereof.

In some embodiments, the JAK1 pathway inhibitor, or a pharmaceutically acceptable salt thereof, is administered in a daily amount of from about 10 mg to about 100 mg on a free base basis. Accordingly, in some embodiments, the selective JAK1 pathway inhibitor is administered in a daily amount of about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg on a free base basis. In some embodiments, the JAK1 pathway inhibitor, or a pharmaceutically acceptable salt thereof, is administered in a daily amount of from about 1 mg to about 100 mg on a free base basis. In some embodiments, the JAK1 pathway inhibitor, or a pharmaceutically acceptable salt thereof, is administered in a daily amount of from about 10 mg to about 80 mg on a free base basis. In some embodiments, the JAK1 pathway inhibitor, or a pharmaceutically acceptable salt thereof, is administered in a daily amount of about 90 mg on a free base basis. In some embodiments, the JAK1 pathway inhibitor, or a pharmaceutically acceptable salt thereof, is administered in a daily amount of about 75 mg on a free base basis. In some embodiments, the JAK1 pathway inhibitor, or a pharmaceutically acceptable salt thereof, is administered in a daily amount of about 45 mg on a free base basis.

The term “about” means “approximately” (e.g., plus or minus approximately 10% of the indicated value).

In some embodiments, the JAK1 pathway inhibitor, or a pharmaceutically acceptable salt thereof, is administered as one or more sustained release dosage forms each comprising the JAK1 pathway inhibitor, or a pharmaceutically acceptable salt thereof.

In some embodiments, the JAK1 pathway inhibitor, or a pharmaceutically acceptable salt thereof, is administered orally.

The embodiments described herein are intended to be combined in any suitable combination as if the embodiments are multiply dependent claims (e.g., the embodiments related to the selective JAK1 pathway inhibitor and doses of the same, the embodiments related to any salt forms of the compounds disclosed herein, the embodiments related to the individual types of cytokine related diseases or disorders, and the embodiments related to composition and/or administration can be combined in any combination).

All possible combinations are not separately listed herein merely for the sake of brevity.

The compounds described herein can be asymmetric (e.g., having one or more stereocenters). All stereoisomers, such as enantiomers and diastereomers, are intended unless otherwise indicated. Compounds that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

In some embodiments, the compound has the (R)-configuration. In some embodiments, the compound has the (S)-configuration.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds described herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds described herein can also include isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆alkyl group of Formulae (I), (II), or (III) or a compound of Table 1 can be optionally substituted with deuterium atoms, such as —CD₃ being substituted for —CH₃). The term, “compound,” as used herein is meant to include all stereoisomers, geometric isomers, tautomers, and isotopes of the structures depicted, unless the name indicates a specific stereoisomer. Compounds herein identified by name or structure as one particular tautomeric form are intended to include other tautomeric forms unless otherwise specified.

All compounds, and pharmaceutically acceptable salts thereof, can be found together with other substances such as water and solvents (e.g. hydrates and solvates) or can be isolated.

In some embodiments, the compounds described herein, or salts thereof, are substantially isolated. By “substantially isolated” is meant that the compound is at least partially or substantially separated from the environment in which it was formed or detected. Partial separation can include, for example, a composition enriched in the compounds described herein. Substantial separation can include compositions containing at least about 50%, at least about 60%, at least about 70%, at least about 80%, at least about 90%, at least about 95%, at least about 97%, or at least about 99% by weight of the compounds described herein, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature” or “rt” as used herein, are understood in the art, and refer generally to a temperature, e.g. a reaction temperature, that is about the temperature of the room in which the reaction is carried out, for example, a temperature from about 20° C. to about 30° C.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “subject”, “individual,” or “patient,” used interchangeably, refers to any animal, including mammals, preferably mice, rats, other rodents, rabbits, dogs, cats, swine, cattle, sheep, horses, or primates, and most preferably humans. In some embodiments, the “subject,” “individual,” or “patient” is in need of said treatment.

In some embodiments, the inhibitors are administered in a therapeutically effective amount. As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the JAK1 inhibitors can prevent prurigo nodularis in an individual who may be predisposed to the disease. The term “preventing” refers to blocking the occurrence of disease in a patient who may be predisposed to the disease but does not yet experience or display the pathology or symptomatology of the disease.

### Combination Therapies

The methods described herein can further comprise administering one or more additional therapeutic agents. The one or more additional therapeutic agents can be administered to a patient simultaneously or sequentially. The one or more additional therapeutic agents can be administered using different methods (e.g., topically).

In some embodiments, the additional therapeutic agent is selected from JAK inhibitors. Additional JAK inhibitors may include ATI-50002 (JAK1/3 selective). Additional JAK inhibitors may include PF-06651600 (JAK3 selective). Additional JAK inhibitors may include PF06700841 (JAK1/TYK2 selective). Additional JAK inhibitors may include baricitinib (JAK1/JAK2 selective). Additional JAK inhibitors may include TYK2 selective inhibitors.

In some embodiments, the additional therapeutic agent is selected from antioxidants. Antioxidants may be selected from pseudocatalase, vitamin E, vitamin C, ubiquinone, lipoic acid, Polypodium leucotomos, catalase/superoxide dismutase combination, and Ginkgo biloba. In some embodiments, antioxidants may be further administered in combination with phototherapy. The administration of antioxidants during or before phototherapy aims to counteract the oxidative stress induced by UV radiation itself, increasing the phototherapy effectiveness.

In some embodiments, the additional therapeutic agent includes anti-histamines.

In some embodiments, the additional therapeutic agent is an antimetabolite. Antimetabolites may include 5-fluorouracil.

In some embodiments, the additional therapeutic agent is selected from topical corticosteroids, immunomodulators, calcineurin inhibitors, and phototherapy. In some embodiments, the additional therapies are systemic steroids or immunosuppressants.

In some embodiments, the additional therapeutic agent includes steroids (e.g., orally administered steroids) including systemic steroids. Steroid treatment may include oral steroid minipulse therapy (e.g., using betamethasone and/or dexamethasone).

In some embodiments, topical corticosteroids are selected from augmented betamethasone dipropionate, clobetasol propionate, diflorasone diacetate, halobetasol propionate amcinonide, betamethasone valerate, desoximetasone, diflorasone diacetate, fluocinolone acetonide, halcinonide, and triamcinolone acetonide.

In some embodiments, the additional therapeutic agent includes immunomodulators. Immunomodulators may include anti-IL15 therapy (e.g., AMG 714 monoclonal antibody). Immunomodulators may include anti-IL36 therapy (e.g., imsidolimab and spesolimab). Immunomodulators may include anti-TNFalpha therapy (e.g., etanercept and infliximab).

In some embodiments, immunomodulators are selected from apremilast, crisaborole, afamelanotide, minocycline, zinc, tofacitinib, AMG 714 monoclonal antibodies, imsidolimab, spesolimabcyclosporine, etanercept, infliximab, cyclophosphamide, ciclosporin, methotrexate, and sodium oxo-dihydro-acridinylacetate (ODHAA).

In some embodiments, calcineurin inhibitors are selected from tacrolimus (FK-506) and pimecrolimus.

In some embodiments, phototherapy includes exposure to ultraviolet (e.g., excimer lamps or lasers).

In some embodiments, the additional therapeutic agent is a Janus kinase inhibitor. In some embodiments, the Janus kinase inhibitor is administered topically.

In some embodiments, the additional therapeutic agent is a Neurokinin 1 receptor antagonists (e.g., Aprepitant).

In some embodiments, the additional therapeutic agent include anti-IL-4/IL-13 antibodies. In some embodiments, anti-IL-4/IL-13 antibodies are selected from dupilumab, lebrikizumab and tralokinumab.

In some embodiments, the additional therapeutic agent include anti-IL-5 antibodies. In some embodiments, anti-IL-5 antibodies are selected from benralizumab, mepolizumab, and reslizumab.

In some embodiments, the additional therapeutic agent include anti-IL-31 antibodies. In some embodiments, anti-IL-31 antibodies include nemolizumab.

In some embodiments, the additional therapeutic agent is an IL-6 antagonist or receptor antagonist. In some embodiments, the IL-6 receptor antagonist is tocilizumab.

### Pharmaceutical Formulations and Dosage Forms

When employed as pharmaceuticals, the JAK1 pathway inhibitors or pharmaceutically acceptable salts thereof, can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, foams, liquids and powders. Conventional pharmaceutical carriers, aqueous, powder or oily bases, thickeners and the like may be necessary or desirable.

This invention also includes pharmaceutical compositions which contain, as the active ingredient, the JAK1 pathway inhibitor described herein, or a pharmaceutically acceptable salt thereof, in combination with one or more pharmaceutically acceptable carriers (excipients). In some embodiments, the composition is suitable for topical administration. In making the compositions, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The JAK1 pathway inhibitors may be milled using known milling procedures such as wet milling to obtain a particle size appropriate for tablet formation and for other formulation types. Finely divided (nanoparticulate) preparations of the JAK1 selective inhibitors can be prepared by processes known in the art, e.g., see International App. No. WO 2002/000196.

The compositions can be formulated in a unit dosage form, each dosage containing a set amount of the active ingredient as the free form or a salt form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

Similar dosages may be used of the compounds described herein in the methods and uses of the invention.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present invention can vary according to, for example, the particular use for which the treatment is made, the manner of administration of the compound, the health and condition of the patient, and the judgment of the prescribing physician. The proportion or concentration of a compound described herein in a pharmaceutical composition can vary depending upon a number of factors including dosage, chemical characteristics (e.g., hydrophobicity), and the route of administration. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the invention can further include one or more additional pharmaceutical agents such as a chemotherapeutic, steroid, anti-inflammatory compound, or immunosuppressant, examples of which are listed herein.

### Kits

The present invention also includes pharmaceutical kits useful, for example, in the treatment and/or prevention of prurigo nodularis, which include one or more containers containing a pharmaceutical composition comprising a therapeutically effective amount of a compound described herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples have been found to be JAK inhibitors according to at least one assay described herein.

### Example A

In vitro JAK Kinase Assay

JAK1 pathway inhibitors that can be used for the treatment of cytokine-related diseases or disorders are tested for inhibitory activity of JAK targets according to the following in vitro assay described in Park et al., Analytical Biochemistry 1999, 269, 94-104. The catalytic domains of human JAK1 (a.a. 837-1142), JAK2 (a.a. 828-1132) and JAK3 (a.a. 781-1124) with an N-terminal His tag are expressed using baculovirus in insect cells and purified. The catalytic activity of JAK1, JAK2 or JAK3 are assayed by measuring the phosphorylation of a biotinylated peptide. The phosphorylated peptide was detected by homogenous time resolved fluorescence (HTRF). IC₅₀s of compounds are measured for each kinase in the 40 microL reactions that contain the enzyme, ATP and 500 nM peptide in 50 mM Tris (pH 7.8) buffer with 100 mM NaCl, 5 mM DTT, and 0.1 mg/mL (0.01%) BSA. For the 1 mM IC₅₀ measurements, ATP concentration in the reactions is 1 mM. Reactions are carried out at room temperature for 1 hour and then stopped with 20 μL 45 mM EDTA, 300 nM SA-APC, 6 nM Eu-Py20 in assay buffer (Perkin Elmer, Boston, MA). Binding to the Europium labeled antibody takes place for 40 minutes and HTRF signal was measured on a Fusion plate reader (Perkin Elmer, Boston, MA). The compounds in Table 1 were tested in this assay and shown to have the IC50 values also found in Table 1.

### Example B

### JAK1 Mediated Pharmacological Inhibition of PN Pathophysiology

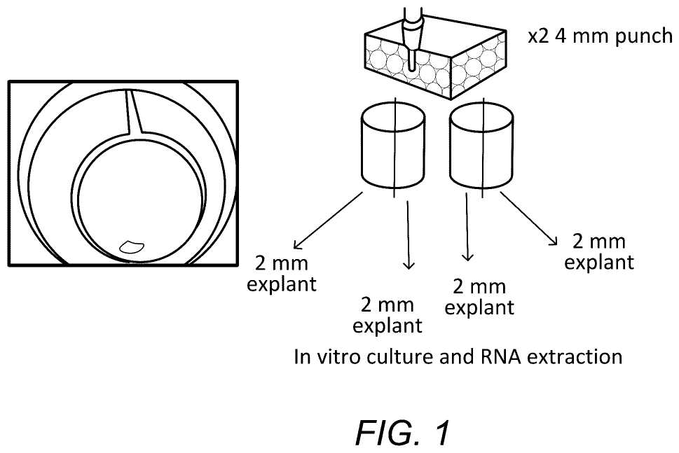

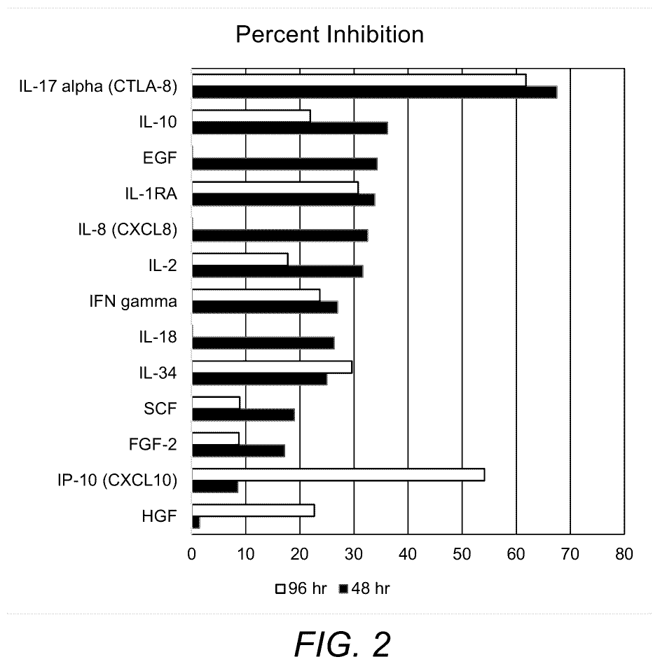

Full-thickness (4 mm) cutaneous skin biopsies of active disease, untreated, prurigo nodularis were obtained. Two (2) 4-mm punch biopsies were obtained for each lesion (see FIG. 1). The biopsies were longitudinally divided into two pieces for a total of four (4) 2-mm full thickness sections, which were then placed in culture. FIG. 1 depicts a representation of skin punch biopsies used for the JAK1 mediated pharmacological inhibition of PN pathophysiology. Biopsies were then cultured in the presence of a vehicle control (DMSO) or small molecule JAK1 inhibitor compound (in this particular case Compound 1) for 8 days. Every 2 days the supernatant was collected for cytokine/protein analysis. Collected supernatants were analyzed to quantify chemokines, cytokines, and growth factors secreted by skin explants in culture. A total of 51 proteins were detected and quantified in culture supernatants by Procarta Multiplex Immunoassay (Thermo Fisher, Waltham, MA). Supernatants and standards were incubated at 4° C. overnight. Assay plates were read on a Luminex 200 Instrument (Luminex Corporation, Austin, TX). The concentration was extrapolated from the antigen standard curve of each analyte. The percent inhibition was determined for each analyte as the difference in protein concentration in cultures with DMSO (C_{D}) versus those with the JAK1 inhibitor, Compound 1 (C_{J}), or (C_{D}-C_{J})/C_{D}, See FIG. 2.

Compound 1 was shown to inhibit key chemokines, cytokines, and growth factors involved in key disease pathways (see Table 2 and FIG. 2). These key chemokines, cytokines, and growth factors are involved in inflammation, pruritus (itching), or lesion development that can be associated with PN. For example, PN lesional skin has been shown to have increased levels of CXCL8, CXCL 10, and interferon-gamma when compared to non-lesional control skin (Tsoi et al., J Allergy Clin Immunol 149, 1329-39 (2022)).

Further, other cytokines, such as IL-31 and IL-17, have also been shown to play a role in PN. For example, CD4+ T-cells has been shown to significantly infiltrate lesional PN skin as compared to non-lesional control skin (Wong, et al., J Investigative Dermatology, 140 (3), 702-706.e2 (2020)). CD4+ T-cells can express interleukin 31 (IL-31), which has been shown to be a mediator of pruritus-an important symptom of PN (Ständer, et al., N Engl J Med, 382:706-716 (2020)). The expressed IL-31 has also been shown to play a role in the development of PN with inhibition of IL-31 by nemolizumab resulting in improvement in pruritus and skin lesions in PN patients (Ständer, supra). When PN skin was cultured nemolizumab (an IL-31 inhibitor), a reduction in IL-17 levels in lesional skin was observed, suggesting that inhibiting IL31R signaling can impact IL-17 expression (Tsoi, supra). Further, cells expressing IL-17 are increased in the dermis of PN lesional skin as compared to control skin (Wong, supra). Further, IL-2 is critical to the development, maintenance and function of the infiltrating CD4+ T-cells (Furtado, J Exp Med, 196 (6): 851-7 (2002)) and is a strong itch mediator (Xie, et al., J Dermatol, 46 (3), 177-185 2019)). Further, the epidermis of PN skin has been shown to be positive for STAT6, a marker for Th2 cytokines, such as IL-5 and IL-31 (Mullins, et al., NLM, StatPearls Publishing, https://www.ncbi.nlm.nih.gov/books/NBK459204), Sep. 14, 2021)). Finally, keratinocytes are implicated in the pathogenesis of PN, particularly around pruritus (Zhong et al, Acta Dermato-Venereologicavolume 99, 579-5861 May (2019)). In skin, keratinocytes produce IL-34.

Hence, Compound 1 has been shown to inhibit multiple key chemokines, cytokines, and growth factors involved in PN and/or the underlying inflammation and pruritus associated with PN.

TABLE 2
showing percent inhibition due to Compound 1

| | | |
|---|---|---|
| HGF | 1.46 | 22.65 |
| IP-10 (CXCL10) | 8.53 | 54.12 |
| IL-22 | 11.16 | ** |
| VEGF-A | 16.92 | 7.79 |
| FGF-2 | 17.17 | 8.70 |
| SCF | 18.98 | 8.85 |
| IL-5 | 20.25 | ** |
| IL-34 | 24.98 | 29.61 |
| IL-18 | 26.35 | ** |
| IFN GAMMA | 26.98 | 23.67 |
| IL-2 | 31.63 | 17.75 |
| IL-8 (CXCL8) | 32.51 | 4.38 |
| IL-1RA | 33.82 | 30.75 |
| EGF | 34.30 | ** |
| IL-10 | 36.18 | 21.91 |
| IL-17 ALPHA (CTLA-8) | 67.51 | 61.79 |

**no measureable inhibition

### Example C

### Phase 2 Study of Compound 1

### Study Design

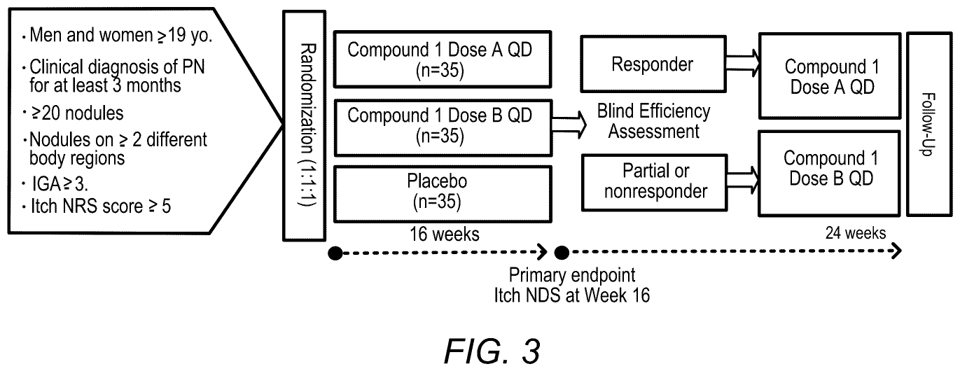

A phase 2 randomized, double-blind, placebo-controlled dose-ranging study of the efficacy and safety of JAK inhibitor Compound 1 treatment of prurigo nodularis patients is described. FIG. 3 depicts the study conducted using Compound 1 for treating participants with prurigo nodularis. The study can include a 24-week double-blind extension period in participants with prurigo nodularis. Participants can include those with prurigo nodularis (e.g., clinically diagnosed for at least 3 months) who have a total of ≥20 nodules, nodules on ≥2 different body regions, an IGA ≥3, and severe pruritus. The study can include men and women of at least 18 years of age.

Participants will participate for up to 48 weeks, including up to 4 weeks for screening, up to 40 weeks for treatment (16 weeks in the placebo-controlled period and 24 weeks in the extension), and 4 weeks for safety follow-up. Approximately 105 participants will be randomized 1:1:1 to 1 of 3 treatment groups (Dose A (45 mg (e.g., 3 Compound 1 15 mg tablets and 2 placebo tablets)), Dose B (75 mg (e.g., 5 Compound 1 15 mg tablets)), or placebo (e.g., 5 placebo tablets)). Compound 1 or matching placebo will be taken orally QD. Based on the efficacy response at Week 16, participants will receive 1 of the 2 doses of active study drug for an additional 24 weeks. For example, someone is a responder if responsive to treatment by achieving ≥4-point decrease in Itch NRS (based on weekly average itch score) and IGA-TS (score of 0 or 1 with ≥2 grade improvement from baseline) and not responsive if they do not meeting the definition of a responder.

In some embodiments, criteria for participants to be included in the study include any one of the following: men and women ≥18 years of age or above; clinical diagnosis of prurigo nodularis for at least 3 months; ≥20 nodules; nodules on ≥2 different body regions; IGA ≥3; and severe pruritus. Severe pruritus may be defined by: an average Itch NRS score ≥5 for the week preceding screening; and an average Itch NRS ≥5 for the week before Day 1 (note: participants must have at least 4 of 7 days of itch data before baseline to calculate an average and be randomized). Participants must be willing to take appropriate, medically acceptable, contraceptive measures to avoid pregnancy or fathering a child for the duration of study participation. Also, female participants must have a negative serum pregnancy test at screening and a negative urine pregnancy test before randomization on Day 1.

Embodiments of the schedules of activities for the placebo-controlled and extension periods are presented in Table 3 and Table 4, respectively.

TABLE 3

| | | | | | | | | | Safety | |
|---|---|---|---|---|---|---|---|---|---|---|
| | | | | | | | | | Follow- | Notes |
| | | | | | | | | | up | Week 16 |
| | | Placebo-Controlled Treatment | | | | | | | 28 days | assessments must be |
| | | | Week | | Week | | | | (+7 d) | complete before |
| | Screening | Day | 2 | Week | 6 | Week | Week | Week | after | participant can |
| Visit Day | Days −28 | 1 | (remote) | 4 | (remote) | 8 | 12 | 16/ET | last | continue in the |
| (Range) | to −1 | (Baseline) | (±3 d) | (±3 d) | (±3 d) | (±3 d) | (±3 d) | (±3 d) | dose | study. |

Administrative procedures

| | | | | | | | | | | |
|---|---|---|---|---|---|---|---|---|---|---|
| Informed consent | X | | | | | | | | | |
| I/E criteria review | X | X | | | | | | | | |
| Demography and | X | | | | | | | | | General and disease- |
| medical history | | | | | | | | | | specific medical |
| | | | | | | | | | | history. |
| Prior/concomitant | X | X | X | X | X | X | X | X | X | |
| medications | | | | | | | | | | |
| Contact IRT | X | X | | X | | X | X | X | | |
| Randomization | | X | | | | | | X* | | * Possible change in |
| | | | | | | | | | | dose based on |
| | | | | | | | | | | efficacy assessment. |
| Distribute | | X | | X | | X | X | X | | |
| reminder card and | | | | | | | | | | |
| diaries | | | | | | | | | | |
| Study drug taken | | | | | | | | | | |
| at visit | | | | | | | | | | |
| Dispense study | | X | | X | | X | X | X | | |
| drug | | | | | | | | | | |
| Collect study drug | | X | | X | | X | X | X | | |
| and review diary | | | | | | | | | | |
| cards | | | | | | | | | | |
| Assess e-diary | | X | | X | | X | X | X | | |
| compliance | | | | | | | | | | |
| Assess study drug | | X | | X | | X | X | X | | |
| compliance | | | | | | | | | | |

Safety assessments

| | | | | | | | | | | |
|---|---|---|---|---|---|---|---|---|---|---|
| AE assessments | X | X | X | X | X | X | X | X | X | If an AE is noted, that |
| | | | | | | | | | | body systems should |
| | | | | | | | | | | be physically |
| | | | | | | | | | | examined. |
| Comprehensive | X | | | | | | | X* | | *Comprehensive |
| physical | | | | | | | | | | physical examination |
| examination | | | | | | | | | | only if ET visit. |
| Height | X | | | | | | | | | |
| Body weight | X | | | | | | | X | | |
| Vital signs | X | X | | X | | X | X | X | X | |
| 12-lead ECG | X | | | | | | | X | | |

Efficacy assessments

| IGA | X | X | X | X | X | X | X |
|---|---|---|---|---|---|---|---|
| Nodule | X | X | X | X | X | X | X |
| assessment | | | | | | | |
| Itch NRS | Diary is completed each evening from screening through the dose of | | | | | | |
| | study drug. | | | | | | |
| Photography | | X | | X | | X | X |

Quality of life assessments Note: Should be conducted before any other assessments.

| | | | | | | | |
|---|---|---|---|---|---|---|---|
| DLQI | X | X | X | X | X | X | X |
| EQ-5D | X | X | X | X | X | X | X |
| PGIC | | | X | X | X | X | X |
| HADS | X | X | X | X | X | X | X |
| FACIT Fatigue | X | X | X | X | X | X | X |
| PROMIS Sleep | X | X | X | X | X | X | X |

Laboratory assessments Note: Blood draw for clinical laboratory tests must be performed before study drug is taken in-
clinic, as appropriate.

| | | | | | | | | | | |
|---|---|---|---|---|---|---|---|---|---|---|
| Chemistry | X | X* | X | X | X | X | X | X | X | *Not necessary if |
| assessments | | | | | | | | | | screening assessment |
| | | | | | | | | | | performed within 14 |
| | | | | | | | | | | days of Day 1. |
| Hematology | X | X* | X | X | X | X | X | X | X | |
| assessments | | | | | | | | | | |
| Pregnancy testing | X | X | | X | | X | X | X | X | *WOCBP will have a |
| | | | | | | | | | | serum test at |
| | | | | | | | | | | screening and follow- |
| | | | | | | | | | | up and a urine test at |
| | | | | | | | | | | all other in-clinic |
| | | | | | | | | | | visits. A positive |
| | | | | | | | | | | urine test must be |
| | | | | | | | | | | confirmed by a serum |
| | | | | | | | | | | test. |
| Coagulation | X | | | | | X | | X | | |
| Urinalysis | X | | | | | X | | X | | |
| TB screening | X | | | | | | | | | |
| Serology | X | | | | | | | | | |
| Thyroid function | X | | | | | | | | | |
| Inflammation | | X | | | | X | | X | | |
| marker | | | | | | | | | | |
| PK blood draw | | | | X | | | X | | | Visits TBD |
| Skin biopsy | | | | | | | | | | Visits TBD |

TABLE 4

| | | | | | | | | | Safety | |
|---|---|---|---|---|---|---|---|---|---|---|
| | | | | | | | | | Follow- | |
| | | | | | | | | | up | |
| | Extension Treatment | | | | | | | | 28 days | |
| | Week | | Week | | | | | | (+7 d) | |
| | 18 | Week | 22 | Week | Week | Week | Week | Week | after | |
| Visit Day | (remote) | 20 | (remote) | 24 | 28 | 32 | 36 | 40/ET | last | |
| (Range) | (±3 d) | (±3 d) | (±3 d) | (±3 d) | (±3 d) | (±3 d) | (±3 d) | (±3 d) | dose | Notes |

Administrative procedures

| | | | | | | | | | |
|---|---|---|---|---|---|---|---|---|---|
| Prior/concomitant | X | X | X | X | X | X | X | X | X |
| medications | | | | | | | | | |
| Contact IRT | | X | | X | X | X | X | X | |
| Distribute | | X | | X | X | X | X | X | |
| reminder | | | | | | | | | |
| cards | | | | | | | | | |
| Study drug | | | | | | | | | |
| taken at visit | | | | | | | | | |
| Dispense | | X | | X | X | X | X | | |
| study drug | | | | | | | | | |
| Collect study | | X | | X | X | X | X | X | |
| drug and | | | | | | | | | |
| review diaries | | | | | | | | | |
| Assess e-diary | | X | | X | X | X | X | X | |
| compliance | | | | | | | | | |
| Assess study | | X | | X | X | X | X | X | |
| drug | | | | | | | | | |
| compliance | | | | | | | | | |

Safety assessments

| | | | | | | | | | | |
|---|---|---|---|---|---|---|---|---|---|---|
| AE | X | X | X | X | X | X | X | X | X | If an AE is noted, |
| assessments | | | | | | | | | | that body systems |
| | | | | | | | | | | should be physically |
| | | | | | | | | | | examined. |
| Comprehensive physical | | | | | | | | X | | |
| examination | | | | | | | | | | |
| Body weight | | | | | X | | | X | | |
| Vital signs | | X | | X | X | X | X | X | X | |
| 12-lead ECG | | | | | | | | X | | |

Efficacy assessments

| IGA | | X | X | X | X | X | X | X | |
|---|---|---|---|---|---|---|---|---|---|
| Itch NRS | Diary is completed each evening from screening through the dose of | | | | | | | | |
| | study drug. | | | | | | | | |
| Nodule | | X | X | X | X | X | X | X | |
| assessment | | | | | | | | | |
| Photography | | | | | | | | | Maybe not at all in |
| | | | | | | | | | the ext. |

Quality of life assessments Note: Should be conducted before any other assessments.

| | | | | | | | |
|---|---|---|---|---|---|---|---|
| DLQI | X | X | X | X | X | X | X |
| EQ-5D-5L | X | X | X | X | X | X | X |
| PGIC | X | X | X | X | X | X | X |
| HADS | X | X | X | X | X | X | X |
| FACIT- | X | X | X | X | X | X | X |
| Fatigue | | | | | | | |
| PROMIS | X | X | X | X | X | X | X |
| Sleep | | | | | | | |

Laboratory assessments Note: Blood draw for clinical laboratory tests must be performed before study drug is taken in-
clinic, as appropriate.

| | | | | | | | | | | |
|---|---|---|---|---|---|---|---|---|---|---|
| Chemistry | X | X | X | X | X | X | X | X | X | |
| Hematology | X | X | X | X | X | X | X | X | X | |
| Pregnancy | | X | | X | X | X | X | X | | *WOCBP: serum test |
| | | | | | | | | | | at screening and |
| | | | | | | | | | | follow-up and urine |
| | | | | | | | | | | test at all other in- |
| | | | | | | | | | | clinic visits. Positive |
| | | | | | | | | | | urine test must be |
| | | | | | | | | | | confirmed by a serum |
| | | | | | | | | | | test. |
| Coagulation | | | | X | | | | X | | |
| Urinalysis | | | | X | | | | X | | |
| Thyroid | | | | | | | | | | |
| function | | | | | | | | | | |
| Inflammation | | | | X | | | | X | | |
| marker | | | | | | | | | | |
| Pharmacokinetics | | | | X | | | | | | Visits TBD |

In some embodiments, criteria for participants to be excluded from the study include any one of the following: chronic pruritus due to a condition other than prurigo nodularis (e.g., such as scabies, insect bite, lichen simplex chronicus, psoriasis, acne, folliculitis, habitual picking, lymphomatoid papulosis, chronic actinic dermatitis, dermatitis herpetiformis, sporotrichosis, bullous disease, excoriation syndrome); neuropathic and psychogenic pruritus (e.g., notalgia paresthetica, brachioradial pruritus, small fiber neuropathy, skin picking syndrome, or delusional parasitosis); and concurrent conditions and history of other diseases. Concurrent conditions and history of other diseases can include: thrombocytopenia, coagulopathy, platelet dysfunction, or history of thrombotic events; immunocompromised (e.g., lymphoma, acquired immunodeficiency syndrome, Wiskott-Aldrich Syndrome) or have a history of malignant disease within 5 years before baseline; chronic or acute infection requiring treatment with systemic antibiotics, antivirals, antiparasitics, antiprotozoals, or antifungals within 2 weeks before the baseline visit; active acute bacterial, fungal, or viral skin infection (e.g., herpes simplex, herpes zoster, chicken pox, clinically infected atopic dermatitis, impetigo) within 1 week before the baseline visit; any other concomitant skin disorder (e.g., generalized erythroderma such as Netherton's Syndrome), pigmentation, or extensive scarring that in the opinion of the investigator may interfere with the evaluation of PN lesions or assessments of efficacy or compromise participant safety; current herpes zoster infection, a history of disseminated herpes simplex, or a history of herpes zoster; history of malignancy, including melanoma, lymphoma, and leukemia within 5 years before Day 1, other than a successfully treated nonmetastatic cutaneous squamous cell carcinoma, basal cell carcinoma, or localized carcinoma in situ of the cervix; and albinism.

In some embodiments, criteria for participants to be excluded any of the following treatments within an indicated washout period before the baseline visit: current use of anticoagulants or medications known to cause thrombocytopenia; 4 weeks—systemic corticosteroids or adrenocorticotropic hormone analogs, cyclosporin, methotrexate, azathioprine, or other systemic immunosuppressive or immunomodulating agents (e.g., mycophenolate or tacrolimus) (note: use of corticosteroid inhalers and intranasal sprays is allowed and use of oral corticosteroids for nondermatologic conditions (e.g., asthma exacerbation, bronchitis) is allowed for no longer than 7 days, if deemed acceptable by the investigator and the sponsor); 2 weeks—systemic antibiotics and immunizations; sedating antihistamines unless on long-term stable regimen (nonsedating antihistamines are permitted) (note: live vaccine are not recommended during the course of the study); 1 week—use of any topical treatments for PN (other than bland emollients), such as corticosteroids, calcineurin inhibitors, PDE4 inhibitors, coal tar (shampoo), topical antibiotics, antibacterial cleansing body wash/soap (unless on long-term stable regimen); <12 weeks or 5 half-lives (if known), whichever is longer, for any topical or systemic JAK or TYK2 inhibitor; <12 weeks or 5 half-lives (if known), whichever is longer, for any investigational or experimental treatments; <12 weeks or 5 half-lives (if known), whichever is longer, for systemic immunosuppressive or immunomodulating biologic drugs; <6 weeks for live vaccine, or planning to receive live vaccine during the course of the study or within 6 weeks after the last dose of study drug; <3 weeks for any oral or topical PDE-4 inhibitor (e.g., apremilast, crisaborole); <2 weeks or 5 half-lives (if known), whichever is longer, for strong and moderate systemic CYP3A4 inhibitors and strong systemic CYP3A4 inducers; and <1 week for antiplatelet drugs, note: Low-dose acetylsalicylic acid (≤100 mg QD) is permitted for the purpose of cardiovascular prophylaxis at the discretion of the investigator.

In some embodiments, criteria for participants to be excluded from the study include uncontrolled thyroid function at screening as determined by the investigator (note: if the participant has a history of thyroid disease and is on treatment, the participant must be on a stable thyroid regimen for at least 3 months prior to Day 1). In some embodiments, criteria for participants to be excluded from the study include laboratory values at screening defined in Table 5.

TABLE 5

| | |
|---|---|
| Laboratory Parameter | Exclusion Criterion |

Hematology

| | | |
|---|---|---|
| a | Platelets | <150 × 10⁹/L |
| b | Hemoglobin | <10 g/L |
| c | ANC | <1.5 × 10⁹/L |
| d | WBC | ≤3.0 × 10⁹/L |

Hepatic

| | | |
|---|---|---|
| e | ALT | ≥2 × ULN |
| f | AST | ≥2 × ULN |
| g | Conjugated (direct) | ≥1.2 × ULN/≥1.5 × ULN |
| | bilirubin/total | (Note: unless clinical diagnosis |
| | bilirubin | of Gilbert's syndrome) |
| h | Alkaline phosphatase | ≥2 × ULN |

Renal

| | | |
|---|---|---|
| j | eGFR | ≤70 mL/minute based on Cockcroft- |
| | | Gault formula |

Coagulation

| | | |
|---|---|---|
| l | PT | >ULN |
| m | INR | >ULN |

In some embodiments, criteria for participants to be excluded from the study include evidence of HBV or HCV infection or risk of reactivation. Participants cannot be positive for hepatitis B surface antigen, anti-hepatitis B core antibody, or HCV antibody; participants also cannot be positive for HBV DNA or HCV RNA in case these reflexive assessments are required to be performed (participants with no prior history of HBV infection who have been vaccinated against HBV and who have a positive antibody (hepatitis B surface antibody) against HBsAg as the only evidence of prior exposure may participate in the study; and participants with a history of HCV infection who are antibody positive and have been successfully treated >12 weeks ago, and have no detectable HCV RNA, are allowed in the study). In some embodiments, criteria for participants to be excluded from the study include known HIV infection.

In some embodiments, criteria for participants to be excluded from the study include evidence of active or latent or inadequately treated infection with Mycobacterium tuberculosis (i.e., TB) as defined by the following: a positive QFT-GIT or positive Mantoux/PPD tuberculin skin test performed at or within the 12 weeks prior to Day 1 is exclusionary; a history of either untreated or inadequately treated latent or active TB infection; if a participant has previously received an adequate course of therapy for either latent TB infection or active TB infection, neither a QFT-GIT nor a Mantoux/PPD tuberculin skin test is needed, but a chest x-ray(s) or other appropriate diagnostic image, performed within 3 months of Day 1, is required; and a participant who is currently being treated for active TB infection is to be excluded.

In some embodiments, criteria for participants to be excluded from the study include known hypersensitivity or severe reaction to Compound 1 or excipients of Compound 1. In some embodiments, criteria for participants to be excluded from the study include pregnant or lactating participants, or those considering pregnancy. In some embodiments, criteria for participants to be excluded from the study include history of alcoholism or drug addiction within 1 year before screening or current alcohol or drug use that, in the opinion of the investigator, will interfere with the participant's ability to comply with the administration schedule and study assessments. In some embodiments, criteria for participants to be excluded from the study include inability or unlikeliness of the participant to comply with the dose schedule and study evaluations, in the opinion of the investigator.

In some embodiments, efficacy of the treatment method disclosed herein can be established based upon an Investigator's Global Assessment (IGA). The IGA is The IGA is an overall severity rating on a 0 to 4 as noted in Table 6. In some embodiments, the IGA-TS (Investigator's Global Assessment Treatment Success) is defined as an IGA score of 0 or 1 with ≥2 grade improvement from baseline. Efficacy may be established if by evaluating a proportion of participants achieving an IGA-TS (IGA of 0 or 1 with a 2-point decrease) at a designated point of time (e.g., week 16).

TABLE 6

| Grade | Severity | Status |
|---|---|---|
| 0 | Clear | No nodules (0 nodules) |
| 1 | Almost | Rare, flattened lesions, with no more than 5 |
| | clear | dome-shaped palpable nodules (approximately |
| | | 1-5 nodules) |
| 2 | Mild | Few, mostly flattened lesions, with small number |
| | | of dome-shaped palpable nodules (approximately |
| | | 6-19 nodules) |
| 3 | Moderate | Many lesions, partially flattened, and dome- |
| | | shaped palpable nodules (approximately 20-100 |
| | | nodules) |
| 4 | Severe | Abundant lesions, majority are dome-shaped |
| | | palpable nodules (over 100 nodules) |

In some embodiments, efficacy of the treatment method disclosed herein can be established based upon Itch Numerical Rating Scale (Itch NRS). Participants may be issued a hand-held device (eDiary) for daily assessments. The participant can be instructed to complete the diary during specific timepoints needed for each assessment beginning on the day of screening through a set endpoint (e.g., Week 40) or treatment discontinuation. In some embodiments, efficacy may be demonstrated by achieving a preset proportion of participants achieving at least 2 or 4-point improvement in Itch NRS (e.g., at Week 16). In some embodiments, efficacy may be demonstrated by observing time to ≥2-point or ≥4-point improvement from baseline in Itch NRS. In some embodiments, Compound 1 and/or methods of use described herein result in an improvement in a participant's response to NRS from baseline.

In some embodiments, efficacy of the treatment method disclosed herein can be established based upon Nodule Assessment. In some embodiments, the number of nodules will be counted at each in-clinic visit and disease activity will be assessed via the PAS. In some embodiments, Compound 1 and/or methods of use described herein result in an improvement in a participant's response to PAS from baseline. PAS (Prurigo Activity Score) will assess disease activity in terms of percentage of pruriginous lesions with excoriations/crusts on top (reflecting active scratching) and the percentage of healed pruriginous lesions is measured by the subitem “Activity” of PAS in order to quantify change of Prurigo nodularis skin lesions.

In some embodiments, efficacy of the treatment method disclosed herein can be established based upon photography of effected areas. Photography of the body areas affected with PN will be obtained at visits. All sites will use 2-dimensional photography to photograph areas of the body containing PN nodules.

In some embodiments, efficacy of the treatment method disclosed herein can be established based upon patient-reported outcomes (PROs). In some embodiments, efficacy of the treatment method disclosed herein can be established based upon a Dermatology Life Quality Index (DLQI). In some embodiments, Compound 1 and/or methods of use described herein result in an improvement in a participant's response to DLQI from baseline. The DLQI is a validated questionnaire (e.g., 10-question) to measure how much the skin problem has affected the participant over the previous 7 days as outlined in the SoAs. The participant will answer the questionnaire with either (1) very much, (2) a lot, (3) a little, or (4) not at all. The questionnaire can be analyzed under 6 headings: symptoms and feelings; daily activities; leisure; work and school; personal relations; and treatment.

In some embodiments, efficacy of the treatment method disclosed herein can be established based upon Patient Global Impression of Change (PGIC). In some embodiments, Compound 1 and/or methods of use described herein result in an improvement in a participant's response to PGIC from baseline. The PGIC is a participants' self-reporting measure that reflects their belief about the efficacy of treatment. The PGIC is a 7-point scale depicting a participant's rating of overall improvement and will be captured during site visits. For example, the participant will answer the following: “Since the start of the treatment you've received in this study, your PN in areas treated with the study drug is: (1) very much improved, (2) much improved, (3) minimally improved, (4) no change, (5) minimally worse, (6) much worse, and (7) very much worse.”

In some embodiments, efficacy can be evaluated based upon improvement in a participant's Hospital Anxiety and Depression Scale (HADS). In some embodiments, Compound 1 and/or methods of use described herein result in an improvement in a participant's response to HADS from baseline. HADS is 14-item questionnaire that assesses the levels of anxiety and depression that a person is currently experiencing. There are 7 questions each for measuring anxiety and for measuring depression, with 4 possible responses to each question (responses are scored as 0, 1, 2, or 3). Separate scores are calculated for anxiety and depression.

In some embodiments, efficacy can be evaluated based upon improvement in a participant's Functional Assessment of Chronic Illness Therapy-Fatigue Scale (FACIT-Fatigue Scale). In some embodiments, Compound 1 and/or methods of use described herein result in an improvement in a participant's response to FACIT-Fatigue Scale from baseline. The FACIT-Fatigue scale is validated instrument, with a 13-item questionnaire originally designed to assess fatigue in patients with chronic disease as a 7-day recall period. Each item uses a 5-point scale ranging from “4=not at all” to “0=very much.” The total FACIT-Fatigue score ranges from 0 (maximum fatigue) to 52 (no fatigue). The participant should complete the questionnaire before site personnel perform any clinic assessments and before any interaction with the site personnel has occurred to avoid biasing the participant's response.

In some embodiments, efficacy can be evaluated based upon improvement in a participant's PROMIS Sleep Scale. In some embodiments, Compound 1, and/or methods of use described herein result in an improvement in a participant's response to PROMIS Sleep Scale from baseline. With the PROMIS Sleep Scale sleep will be assessed using 2 of the PROMIS: Short Forms, Sleep-Related Impairment and Sleep-Disturbance. The PROMIS Short Form—Sleep-Related Impairment questionnaire assesses self-reported perceptions of alertness, sleepiness, and tiredness during usual waking hours and the perceived functional impairments during wakefulness associated with sleep problems or impaired alertness. The questionnaire has 8 questions, which are assessed using a 5-point scale. Scores range from 8 to 40, with higher scores indicating greater severity of sleep-related impairment. The PROMIS Short Form—Sleep Disturbance questionnaire assesses self-reported perceptions of sleep quality, sleep depth, and restoration associated with sleep. Sleep disturbance does not focus on symptoms of specific sleep disorders and does not provide subjective estimates of sleep quantities (e.g., total amount of sleep, time to fall asleep, amount of wakefulness during sleep). The sleep disturbance short form is generic rather than disease-specific. The questionnaire had 8 questions and is assessed using a 5-point scale. Scores range from 8 to 40, with higher scores indicating greater severity of sleep disturbance.

In some embodiments, efficacy can be evaluated based upon improvement in a participant's EQ-5D-5L questionnaire. In some embodiments, Compound 1, and/or methods of use described herein result in an improvement in a participant's response to the EQ-5D-5L questionnaire from baseline. The EQ-5D-5L questionnaire is a standardized, validated instrument for use as a measure of health outcome. The EQ-5D-5L questionnaire will provide data for use in economic models and analyses, including developing health utilities or Quality-Adjusted Life Years (QALYs). The EQ-5D-5L questionnaire consists of the following 2 sections: the EQ-5D descriptive system and the EQ VAS. The descriptive system comprises five dimensions: mobility, self-care, usual activities, pain/discomfort, and anxiety/depression.

Each dimension has 5 levels: Level 1 is “no problems,” Level 2 is “slight problems,” Level 3 is “moderate problems,” Level 4 is “severe problems,” and Level 5 is “extreme problems.” This part of the EQ-5D-5L questionnaire provides a descriptive profile that can be used to generate a health state profile. For example, a participant in “health state 12345” would have no problems with mobility, slight problems with self-care (washing or dressing), moderate problems with doing usual activities, severe pain or discomfort and extreme anxiety or depression. Each health state can potentially be assigned a summary index score based on societal preference weights for the health state. These weights, sometimes referred to as utilities, are often used to compute QALYs for use in health economic analyses. Health state index scores generally range from less than 0 (where 0 is the value of a health state equivalent to dead; negative values representing values as worse than dead) to 1 (the value of full health), with higher scores indicating higher health utility. The health state preferences often represent national or regional values and can therefore differ between countries/regions. The EQ VAS records the participant's self-rated health on a vertical visual analogue scale (0 to 100), where the endpoints are labelled “the best health you can imagine” (100 score) and “the worst health you can imagine” (0 score).

Several populations will be analyzed. In some embodiments, an intent-to-treat (ITT) will be analyzed. The ITT population includes all randomized participants. Treatment groups for this population will be defined according to the treatment assignment at randomization. In some embodiments, a safety population will be analyzed. The safety population includes all participants who received at least 1 dose of study drug, Treatment groups for this population will be determined according to the actual treatment the participant received on Day 1. In some embodiments, a PK/PD evaluable population will be analyzed. The PK/PD evaluable population includes participants who received at least 1 dose of study drug (Compound 1 and/or placebo) and provided at least 1 postdose PK/PD sample/assessment. The study pharmacokineticist will review data listings of participant administration and sample records to identify participants to be excluded from the analysis.

### Statistical Analyses

In some embodiments, a primary analysis will be based on the ITT population. The primary alternative hypothesis (superiority of Compound 1 groups compared with placebo group for proportion of participants achieving 4-point improvement in Itch NRS at Week 16 (responders)) will be tested using exact logistic regression. This model will include the treatment groups and stratification factors. The unadjusted p-values between each of Compound 1 groups versus placebo group will be compared with the pre-specified the significance level. Odds ratio and corresponding 95% confidence interval will be provided as well. All nonresponders in the placebo-controlled period, as well as all participants who are missing post-baseline values, will be defined as nonresponders for the nonresponder imputation analysis.

In some embodiments, a secondary efficacy analyses will be conducted on the ITT population. All secondary efficacy variables will be summarized using descriptive statistics. For the binary endpoint, proportion of participants achieving an IGA-TS (IGA of 0 or 1 with a 2-point decrease) at Week 16, summary statistics will include sample size, frequency, and percentages. For the time-to-event endpoint, time to ≥2-point improvement from baseline in Itch NRS, Kaplan-Meier curves will be presented by treatment groups. The number of participants, number of events and number of censoring will be summarized by treatment groups. The KM estimate of median time will be presented with its 95% CI.

In some embodiments, a safety analyses will be conducted on the safety population. A TEAE is any AE either reported for the first time or worsening of a pre-existing event after first dose of study drug up to 28 days after the last dose of study drug. Analysis of AEs will be limited to TEAEs, but data listings will include all AEs regardless of their timing to study drug administration. Adverse events will be tabulated by the MedDRA preferred term and system organ class. Severity of AEs will be based on the National Cancer Institute CTCAE v5.0 using Grades 1 through 5.

The subset of AEs considered by the investigator to have a relationship to study drug will be considered to be treatment-related AEs. If the investigator does not specify the relationship of the AE to study drug, then the AE will be considered treatment-related. The incidence of AEs and treatment-related AEs will be tabulated.

The clinical laboratory data will be analyzed using summary statistics; no formal treatment group comparisons are planned. Laboratory test values outside the normal range will be assessed for severity based on the normal ranges for the clinical reference laboratory. The incidence of abnormal laboratory values and shift tables relative to baseline will be tabulated. Descriptive statistics and mean change from baseline will be determined for vital signs (blood pressure, pulse, respiratory rate, and body temperature) at each assessment time. Descriptive statistics and mean change from baseline will be determined for each ECG parameters at each assessment time.

In some embodiments, all exploratory efficacy variables will be summarized using descriptive statistics. For categorical measurements, summary statistics will include sample size, frequency, and percentages. For continuous measurements, summary statistics will include sample size, mean, median, standard deviation, minimum, and maximum. Summary statistics for continuous measures will be provided for baseline, the actual measurements at each visit, and the change and percentage change from baseline at each visit, if applicable. For the time-to-event endpoint, the number of participants, number of events and number of censoring will be summarized by treatment groups. The KM estimate of median time will be presented with its 95% CI.

In some embodiments, pharmacokinetic analyses will be performed in the PK evaluable population. The Compound 1 plasma concentration data will be analyzed by a population PK modeling approach. Such data may be combined with data from other studies in the clinical development program to develop or refine population PK models, in which populations of healthy participants, hidradenitis suppurativa participants and/or vitiligo participants will be evaluated, and included into the model if significant, as a covariate. This model may be used to evaluate the effects of intrinsic and extrinsic covariates on the PK of Compound 1 and to determine measures of individual plasma exposures (such as steady-state peak, trough, and/or time-averaged concentrations). A data analysis plan and results of population PK analysis will be reported separately.

In some embodiments, pharmacokinetic/pharmacodynamic analysis will be explored in the PK/PD evaluable population. Clinical response such as platelet count, etc, will be analyzed. A data analysis plan and results of population PK/PD analysis will be reported separately.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A method for treating prurigo nodularis in a subject, said method comprising administering to the subject a therapeutically effective amount of a JAK1 pathway inhibitor 4-[3-(cyanomethyl)-3-(3′,5′-dimethyl-1H, 1′H-4,4′-bipyrazol-1-yl)azetidin-1-yl]-2,5-difluoro-N-[(1S)-2,2,2-trifluoro-1-methylethyl]benzamide, or a pharmaceutically acceptable salt thereof, in combination with a corticosteroid to a subject in need thereof.

2. (canceled)

3. The method of claim 1, wherein the JAK1 pathway inhibitor is a pharmaceutically acceptable salt of 4-[3-(cyanomethyl)-3-(3′,5′-dimethyl-1H, 1′H-4,4′-bipyrazol-1-yl) azetidin-1-yl]-2,5-difluoro-N-[(1S)-2,2,2-trifluoro-1-methylethyl]benzamide, or a pharmaceutically acceptable salt thereof.

4. The method of claim 3, wherein the JAK1 pathway inhibitor is 4-[3-(cyanomethyl)-3-(3′,5′-dimethyl-1H, 1′H-4,4′-bipyrazol-1-yl) azetidin-1-yl]-2,5-difluoro-N-[(1S)-2,2,2-trifluoro-1-methylethyl]benzamide phosphoric acid salt.

5. (canceled)

6. (canceled)

7. The method of claim 1, wherein the JAK1 pathway inhibitor, or pharmaceutically acceptable salt thereof, is administered in a daily dose of about 5 mg to about 95 mg on a free base basis.

8. The method of claim 1, wherein the JAK1 pathway inhibitor, or pharmaceutically acceptable salt thereof, is administered in a daily dose of about 45 mg or about 75 mg on a free base basis.

9. The method of claim 1, wherein the JAK1 pathway inhibitor, or pharmaceutically acceptable salt thereof, is administered in combination with a further therapeutic agent.

10. The method of claim 9, wherein the further therapeutic agent is a neurokinin 1 receptor antagonist.

11. The method of claim 10, wherein the neurokinin 1 receptor antagonist is aprepitant.

12. The method of claim 9, wherein the further therapeutic agent is an anti-IL-4 and/or IL-13 antibody.

13. The method of claim 12, wherein the anti-IL-4 and/or IL-13 antibody is dupilumab, lebrikizumab or tralokinumab.

14. The method of claim 9, wherein the further therapeutic agent is an anti-IL-5 antibody.

15. The method of claim 14, wherein the anti-IL-5 antibody is benralizumab, mepolizumab, or reslizumab.

16. The method of claim 9, wherein the further therapeutic agent is an anti-IL-31 antibody.

17. The method of claim 16, wherein the anti-IL-31 antibody is nemolizumab.

18. The method of claim 1, wherein the administering comprises administering the JAK1 pathway inhibitor, or pharmaceutically acceptable salt thereof, together with at least one pharmaceutically acceptable carrier or excipient.

19. The method of claim 1, wherein the JAK1 pathway inhibitor, or pharmaceutically acceptable salt thereof, is administered in a daily dose of about 10 mg to about 80 mg on a free base basis.

20. The method of claim 1, wherein the JAK1 pathway inhibitor is 4-[3-(cyanomethyl)-3-(3′,5′-dimethyl-1H, 1′H-4,4′-bipyrazol-1-yl) azetidin-1-yl]-2,5-difluoro-N-[(1S)-2,2,2-trifluoro-1-methylethyl]benzamide.

21. The method of claim 1, wherein the JAK1 pathway inhibitor, or pharmaceutically acceptable salt thereof, is administered in a daily dose of about 45 mg to about 75 mg on a free base basis.

22. The method of claim 1, wherein the JAK1 pathway inhibitor, or pharmaceutically acceptable salt thereof, is administered in a daily dose of about 45 mg on a free base basis.

23. The method of claim 1, wherein the JAK1 pathway inhibitor, or pharmaceutically acceptable salt thereof, is administered in a daily dose of about 75 mg on a free base basis.

24. The method of claim 1, wherein the corticosteroid is a topical corticosteroid.

25. The method of claim 24, wherein the topical corticosteroid is selected from augmented betamethasone dipropionate, clobetasol propionate, diflorasone diacetate, halobetasol propionate amcinonide, betamethasone valerate, desoximetasone, diflorasone diacetate, fluocinolone acetonide, halcinonide, and triamcinolone acetonide.
